(19) United States
(12) Patent Application Publication
(10) Pub. No.: US 2023/0263200 A1
(43) Pub. Date: Aug. 24, 2023

(54) METHOD FOR PRODUCING CULTURED MEAT ON BASIS OF CELL SHEET COATING TECHNIQUE, AND CULTURED MEAT PRODUCED THEREBY

(71) Applicant: Industry-Academic Cooperation Foundation, Yonsei University, Seoul (KR)

(72) Inventors: Jinkee HONG, Seoul (KR); Sohyeon PARK, Seoul (KR)

(73) Assignee: Industry-Academic Cooperation Foundation, Yonsei University, Seoul (KR)

(21) Appl. No.: 18/006,446

(22) PCT Filed: Jul. 22, 2021
(86) PCT No.: PCT/KR2021/009506

§ 371 Date: Mar. 30, 2023

(30) Foreign Application Priority Data
Jul. 22, 2020 (KR) 10-2020-0090736

(51) Int. Cl. A23L 13/00; C12N 5/077; C12M 3/00; C12M 1/12; C12M 1/42; A23P 20/20

(52) U.S. Cl. CPC A23L 13/00; C12N 5/0658; C12M 21/08; C12M 25/04; C12M 35/02; A23P 20/20; C12N 2537/10; C12N 2533/54; C12N 2533/80

(57) ABSTRACT

The present disclosure relates to a method for producing cultured meat, the method including: forming a cell sheet by culturing cells usable for producing cultured meat; and culturing the cells in a form in which a nanofilm is formed on a surface of the cell sheet by coating the cell sheet. The present disclosure provides a method for producing cultured meat that implements excellent mechanical strength by protecting a cell layer from external stress and performing stable cell proliferation, and provides cultured meat implementing quality and taste that are improved compared to those of conventional cultured meat by reproducing tissue similar to muscle tissue of an actual animal.

### CROSS-REFERENCE TO RELATED APPLICATIONS

This application is a 35 U.S.C. § 371 national phase of PCT International Application No. PCT/KR2021/009506, filed Jul. 22, 2021, which claims the benefit of priority under 35 U.S.C. § 119 to Korean Patent Application No. 10-2020-0090736, filed Jul. 22, 2020, the contents of which are incorporated herein by reference in its entirety.

### FIELD

The present disclosure relates to a method for producing cultured meat based on a cell sheet coating technique and cultured meat produced using the same.

### BACKGROUND

According to the report of the Food and Agriculture Organization (FAO), the world population is expected to increase from 7.6 billion in 2018 to 9.5 billion in 2050. However, while the yield of crops is expected to decrease due to the earth's abnormal climate such as global warming, the demand for grains for feed is expected to increase, which will lead to an increase in production costs and a reduction in production area. Therefore, livestock products as food resources are expected to become expensive.

Although animal-derived foods such as livestock products are expensive in terms of energy and production, the animal-derived foods have directly contributed to securing human nutrition because they are the best source of high-quality proteins and micronutrients essential for normal growth and health of humans. In particular, essential amino acids are nutrients that must be taken with foods because they are not synthesized in the body or are synthesized in significantly small quantities. Considering the prediction that the demand for animal-derived foods will reach 550 million tons in 2050, doubling the current level, there is a limit to handling proteins required to supply the essential amino acids with traditional livestock production methods.

Meanwhile, as a solution to a meat shortage problem in the future, cultured meat has recently been attracting attention. The cultured meat, also called substitute meat, refers to edible meat that is obtained through cell proliferation using cell engineering by culturing cells of living animals in a laboratory without going through a process of raising livestock. The cultured meat is also called “in vitro meat” or “lab-grown meat” in the sense that it is grown in a test tube, “artificial meat” in the sense that it is synthesized using stem cells by humans rather than in nature, “clean meat” in the sense that it is produced in a clean production facility rather than in a traditional breeding facility, or “bio-artificial muscles (BAMs)” in the sense that muscle fibers constituting cultured meat are cultured.

The idea of the cultured meat was raised quite a long time ago, and in 1932, British Prime Minister Winston Churchill wrote in a book called “Fifty Years Hence” that fifty years hence, we shall escape the absurdity of growing a whole chicken in order to eat the breast or wing by growing these parts separately under a suitable medium. Later, in 1999, Dr. Willem van Eelen of the University of Amsterdam in the Netherlands, who is called the godfather of cultured meat, obtained an international patent for the theory on cultured meat, and in 2002, succeeded in culturing muscle tissue derived from goldfish in a petri dish in the laboratory.

The method for producing cultured meat that is currently mainly used is as follows. A tissue is collected from a living animal, and then stem cells are isolated from the tissue. Thereafter, the isolated stem cells are cultured into myocytes in the laboratory, grown for several weeks, and then subjected to muscle fiber coloring and fat mixing, thereby producing cultured meat. In this case, in the production process, scaffolds are used, and self-organizing methods are also used.

Although the idea of cultured meat began quite a long time ago, animals still need to be slaughtered in a process of collecting tissues from living animals and isolating stem cells from the tissues to produce cultured meat. In addition, the current cultured meat production technique takes a long time to culture stem cells collected from living cells into myocytes, which still keeps the production cost of cultured meat at a high level. Furthermore, the efficiency of proliferation and differentiation of cells is reduced during long-term culture, and as a result, a yield of cells of cultured meat is significantly reduced.

In addition, the appearance and texture of currently produced cultured meat are significantly different from those of real meat. The development of patties in the form of minced meat has begun, and recently, cultured meat has reached the extent of producing a taste and texture similar to those of processed meat, but there is still a limit to inducing customers to buy it.

In order to commercialize cultured meat, a mass production technique and a culture technique that realizes a form similar to an actual muscle tissue so that a taste and texture similar to those of real meat are realized have been demanded.

### RELATED ART DOCUMENT

### Patent Document

U.S. Pat. No. 7,270,829 (Sep. 18, 2007)

### SUMMARY

The present disclosure has been made to solve the above technical problem, and an object of the present disclosure is to provide a method for producing structured cultured meat by forming organized muscle tissue from cells of cultured meat by a self-organizing technique.

Another object of the present disclosure is to provide a method for producing cultured meat differentiating into a form similar to an actual muscle tissue by improving mechanical properties of a cell sheet through coating and protection of a surface of the cell sheet.

Still another object of the present disclosure is to provide a method for producing cultured meat that facilitates formation of three-dimensional muscle tissue without using a polymer support by solving the problem of a reduction in adhesion when cell sheets are stacked in multiple layers.

Still another object of the present disclosure is to create an environment for mass proliferation and differentiation of cells optimized for cultured meat production, in which cell physical properties may be controlled by strongly maintaining cell-to-cell junctions even when cell sheets are stacked in multiple layers.

In one general aspect, a method for producing cultured meat includes: forming a single cell sheet by culturing cells usable for producing cultured meat; obtaining the single cell sheet; forming a nanofilm on a surface of the cell sheet by coating the obtained single cell sheet; forming a multilayer cell sheet by stacking the coated single cell sheets; and forming muscle tissue from the stacked cell sheets.

In the method for producing cultured meat according to an aspect of the present disclosure, the cells usable for producing cultured meat may be mesenchymal stem cells (MSCs), induced pluripotent stem cells (iPSCs), satellite cells, adipocytes, or embryonic stem cells.

In the method for producing cultured meat according to an aspect of the present disclosure, the coating may be performed to form a multilayer nanofilm using one or two or more selected from the group consisting of electrostatic attraction, van der Waals force, hydrophobic bonding, hydrogen bonding, and covalent bonding.

In the method for producing cultured meat according to an aspect of the present disclosure, the nanofilm may be formed by alternately stacking a positively charged material and a negatively charged material.

In the method for producing cultured meat according to an aspect of the present disclosure, the positively charged material may be one or two or more selected from the group consisting of chitosan, starch, collagen, gelatin, fibrinogen, silk fibroin, casein, elastin, laminin, and fibronectin.

In the method for producing cultured meat according to an aspect of the present disclosure, the negatively charged material may be one or two or more selected from the group consisting of hyaluronic acid, alginate, tannic acid, lignin, cellulose, heparin, carrageenan, agar, xanthan gum, gum arabic, glucomannan, carboxymethylcellulose (CMC), and tara gum.

In the method for producing cultured meat according to an aspect of the present disclosure, a thickness of the nanofilm may be 50 to 5,000 nm.

In the method for producing cultured meat according to an aspect of the present disclosure, the method may further include, after the forming of the nanofilm, forming a protective layer.

In the method for producing cultured meat according to an aspect of the present disclosure, in the culturing of the coated cells, the coated cells may be stimulated by an ultrasonic wave, an electric current, an electromagnetic field, a magnetic field, or a combination thereof.

In the method for producing cultured meat according to an aspect of the present disclosure, the method may further include, after the forming of the nanofilm, adding a cell growth factor.

In the method for producing cultured meat according to an aspect of the present disclosure, the method may further include adding a fat and a colorant to the muscle tissue.

In another general aspect, there is provided cultured meat produced by the method for producing cultured meat.

In still another general aspect, a cell culture platform for producing cultured meat includes: a substrate; a porous coating layer in which a positively charged material and a negatively charged material are alternately stacked; and a protective layer.

In an aspect of the present disclosure, the porous coating layer may be formed by crosslinking the positively charged material and the negatively charged material.

In an aspect of the present disclosure, the porous coating layer may contain C-phycocyanin.

The method for producing cultured meat according to the present disclosure provides an environment optimized for cultured meat production by protecting a cell layer from external stress through coating of a surface of a cell sheet and performing stable cell proliferation.

The method for producing cultured meat according to the present disclosure may provide structured cultured meat by forming organized muscle tissue from cells of cultured meat by a self-organizing technique.

In the method for producing cultured meat according to the present disclosure, as mechanical properties of the cell sheet are improved, a differentiation aspect similar to the actual muscle tissue may be exhibited even when the cell sheets are stacked in multiple layers.

In the present disclosure, when the cell sheets are stacked in multiple layers, adhesion between cells is excellently maintained, such that three-dimensional muscle tissue may be easily formed without a polymer support.

The cell culture platform for producing cultured meat according to the present disclosure may be easily applied to a cell culture plate, a growth factor for cell culture is immobilized in the platform, and exposure to a liquid phase is minimized, such that the growth factor may be provided to cells while maintaining its activity, thereby improving cell proliferation. Therefore, an effective delivery to cells may be implemented with a small amount of nutrients, and it is possible to provide an optimal culture environment for producing cultured meat requiring mass proliferation of cells.

### BRIEF DESCRIPTION OF THE DRAWINGS

FIG. 1 illustrates culture processes in a method for producing cultured meat according to (a) Comparative Example 1 and (b) Example 2 of the present disclosure, and is a comparative schematic view showing whether muscle tissue is effectively formed depending on whether or not a cell sheet is coated.

FIG. 2 illustrates graphs showing differences in mechanical strength depending on whether or not a positively charged layer and a negatively charged layer of a cell sheet are alternately stacked according to (a) Comparative Example 1 and (b) Example 2 of the present disclosure.

FIG. 3A is a schematic view briefly showing a production process of a crosslinked porous nanofilm (X-linked (CHI/CMC)) according to Example 1-2 of the present disclosure.

FIG. 3B is a schematic view briefly showing a production process of a cell culture platform for producing cultured meat according to Example 1-3 of the present disclosure.

FIG. 4A is a graph showing results of FT-IR spectral analysis of a crosslinked porous nanofilm (X-linked (CHI/CMC)) according to Experimental Example 1-1 of the present disclosure.

FIG. 4B shows results of comparative analysis of AFM images of a crosslinked porous nanofilm (X-linked (CHI/CMC)) and a non-crosslinked porous nanofilm according to Experimental Example 1-2 of the present disclosure.

FIG. 5 is a graph showing results of preparing 6, 13, and 30 BL films of crosslinked porous nanofilms and evaluating C-PC release properties thereof.

FIG. 6A illustrates a state of a film in which C-phycocyanin is incorporated into a porous structure thereof and a confocal microscope image thereof, in which in the case of the crosslinked film, a clear fluorescence image is observed after incorporation of C-phycocyanin.

FIG. 6B illustrates SEM images of the non-crosslinked film, the crosslinked film, and the film into which C-PC is incorporated.

FIG. 7 is a graph showing results of cell proliferation according to Experimental Example 2 of the present disclosure.

FIG. 8 is a graph showing a comparison of C-phycocyanin release properties in a film with a protective layer and a film without a protective layer.

FIG. 9 is a graph showing results of performing DNA quantitative analysis of a cell sheet according to Experimental Example 4 of the present disclosure.

FIG. 10 is an optical microscope image showing a cell proliferation state of each group according to Experimental Example 2 of the present disclosure.

FIG. 11A is an image showing results of performing H&E staining on a single layer sheet and a four-layer cell sheet according to Experimental Example 4 of the present disclosure, and FIG. 11B is a graph showing a density quantified by a percentage of a stained area.

FIG. 12 illustrates states of cultured meat before and after cooking.

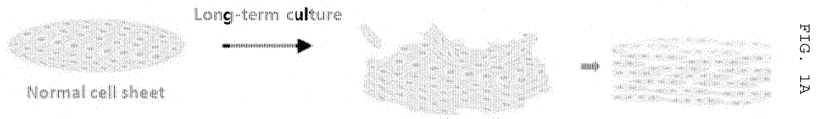

### DETAILED DESCRIPTION

Hereinafter, a method for producing cultured meat according to the present disclosure and cultured meat produced using the same will be described in detail. Here, unless otherwise defined, all the technical terms and scientific terms have the same meanings as commonly understood by those skilled in the art to which the present disclosure pertains. The terms used in the description of the present disclosure are only for effectively describing a certain example rather than limiting the present disclosure.

In addition, a description of the known effects and configurations unnecessarily obscuring the gist of the present disclosure will be omitted in the following description. Hereinafter, a unit used in the present specification without special mention is based on weight, and as an example, a unit of % or a ratio refers to wt % or a weight ratio.

In addition, terms “first”, “second”, A, B, (a), (b), and the like may be used in describing components of the present disclosure. These terms are used only in order to distinguish any component from other components, and features, sequences, or the like of corresponding components are not limited by these terms.

In addition, unless the context clearly indicates otherwise, singular forms used in the specification of the present disclosure may be intended to include plural forms.

Hereinafter, a method for producing cultured meat according to the present disclosure will be described in detail.

A method for producing cultured meat according to the present disclosure includes: forming a single cell sheet by culturing cells usable for producing cultured meat in a culture dish; obtaining the single cell sheet; forming a nanofilm on a surface of the cell sheet by coating the obtained single cell sheet; forming a multilayer cell sheet by stacking the coated single cell sheets; and forming muscle tissue from the stacked cell sheets.

Examples of the cells usable for producing cultured meat include mesenchymal stem cells (MSCs), induced pluripotent stem cells (iPSCs), satellite cells, adipose-derived stem cells (ASCs), and embryonic stem cells.

Each step will be described in more detail below.

First, a tissue is collected from a living animal, and then stem cells that may be used for producing the cultured meat are isolated from the tissue. Since a known stem cell extraction method may be applied to the stem cell extraction method, a detailed description thereof will be omitted. After culturing the stem cells by a cell culture method known in the art, when a single layer cell bundle is formed at a high density enough to cover the entire surface of the culture dish, a low-temperature treatment at 32° C. or lower is performed to induce non-adhesiveness of the cells, and the cell bundle is separated from the culture dish. Alternatively, the single cell bundle is separated from the culture dish by a cell scraper. In this case, the single layer cell bundle may be referred to as a single cell sheet in the specification of the present disclosure.

In the present specification, specifically, the single cell sheet refers to a state where one or more cells are arranged in a single layer in a plate shape, and may be separated from the culture dish by a physical or chemical method as described above.

Thereafter, the surface of the obtained single layer cell sheet is coated to form a nanofilm. The coating may be performed using one or two or more selected from the group consisting of electrostatic attraction, van der Waals force, hydrophobic bonding, hydrogen bonding, and covalent bonding, thereby forming a multilayer nanofilm.

In a preferred embodiment of the present disclosure, the nanofilm may be formed by alternately stacking a positively charged material and a negatively charged material. Specifically, the method includes, after forming the cell sheet by culturing the cells usable for producing cultured meat, alternately immersing the cell sheet in two oppositely charged coating solutions including a first coating solution containing a positively charged material and a second coating solution containing a negatively charged material. The cell sheet is immersed in the first coating solution to introduce a positively charged layer into a cell membrane surface, and then the cell sheet is immersed in the second coating solution to stack a negatively charged layer on the positively charged layer, such that a layer-by-layer (LBL) assembly is performed on a surface of the cell sheet, and a multilayer nanofilm is formed by repeating this operation n times.

The oppositely charged layers obtained by alternate bonding of the positively charged layer and the negatively charged layer may stably maintain bonding through electrostatic attraction, a bonding force between the cell sheets is further enhanced according to an interaction of the positively charged material and the negatively charged material by hydrogen bonding, and the multilayer nanofilm surrounds the cell sheet, such that the cells may be protected in a stable state for a long period of time.

In an embodiment of the present disclosure, a thickness of the nanofilm may be 5 to 5,000 nm. The thickness of the nanofilm may be controlled according to a desired application, and it is preferable that the thickness of the nanofilm is within the above range so as not to act as a barrier to material diffusion as a dense layer is formed on the cells. Preferably, when the thickness of the nanofilm is 10 to 4,000 nm, continuous release of the cell growth factor may be induced. The nanofilm may have two or more layers, and preferably, may have 4 to 40 layers.

In an embodiment of the present disclosure, if necessary, a cleaning process may be further included between introduction of the positively charged layer and then introduction of the next negatively charged layer, or between introduction of the negatively charged layer and then introduction of the next positively charged layer, within a range that does not impair achievement of the object of the present disclosure. The cleaning process refers to a step for removing the materials stacked on the surface of the cell sheet or the charged layer with weak bonding, and may be performed using the same solvent as the first coating solution or the second coating solution. Through the cleaning process, it is possible to achieve an effect of evenly and rapidly forming a coating layer on the surface of the cell sheet.

The first coating solution or the second coating solution may additionally contain a plurality of growth factors required for cell culture, for example, EGF, IGF-1, PDGF, TGF-β, VEGF, bFGF, and the like.

The positively charged material and the negatively charged material should be edible for the production of cultured meat, and are preferably biocompatible organic polymers or inorganic materials.

In an embodiment of the present disclosure, as a specific example, the organic polymer may be one or two or more selected from the group consisting of chitosan, chitin, starch, collagen, gelatin, fibrinogen, silk fibroin, casein, elastin, laminin, and fibronectin. Preferably, the organic polymer may be chitosan, collagen, gelatin, or laminin, but is not particularly limited thereto, as long as it is a cationic polysaccharide polymer. The negatively charged material may be one or two or more selected from the group consisting of hyaluronic acid, alginate, pectin, tannic acid, lignin, cellulose, heparin, carrageenan, agar, xanthan gum, gum arabic, glucomannan, carboxymethylcellulose (CMC), and tara gum. Preferably, the negatively charged material may be carboxymethylcellulose, carrageenan, xanthan gum, or agar, but is not particularly limited thereto, as long as it is an anionic polysaccharide polymer.

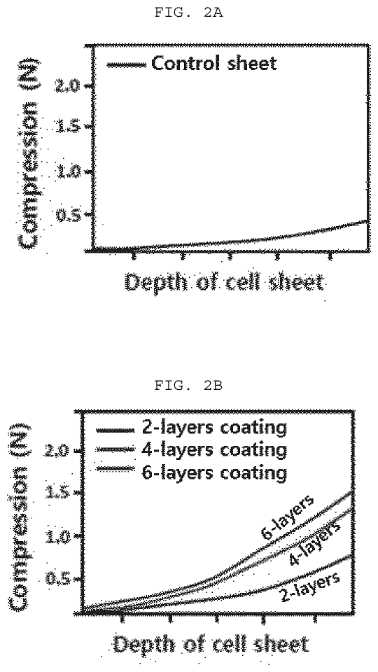

Referring to FIG. 3A, the forming of the nanofilm may be described.

As illustrated in FIG. 3A, a positively charged polysaccharide and a negatively charged polysaccharide may be alternately stacked to form an LbL assembled nanofilm. The polysaccharide is a natural polymer having a functional group capable of forming a hydrogen bond, and preferably includes an NH₂ functional group in a case of a positively charged polysaccharide and a COOH functional group in a case of a negatively charged polysaccharide. As a specific example, in an aqueous solution with a pH of 4 to a pH of 5, NH₂ is converted into NH₃⁺ and COOH is converted into COO⁻, and a positively charged polysaccharide and a negatively charged polysaccharide are alternately stacked through an electrostatic interaction to form an LbL assembled multilayer film.

In this case, in addition to the LbL assembly by the electrostatic interaction, the method may further include inducing crosslinking between the polysaccharide polymer layers. The crosslinking is induced by a crosslinking agent, and as a specific example, ethyl(dimethylaminopropyl)carbodiimide (EDC)/hydroxysuccinimide (NHS) may be used. A first crosslinking may be performed by forming a stable amide bond between an ester of the negatively charged polysaccharide and an amine of the positively charged polysaccharide using the EDC/NHS principle. Alternatively, a reactive end of glutaraldehyde may be induced to form a covalent bond between a hydroxyl group and a primary amine group of the polysaccharide by using glutaraldehyde, thereby further performing a second crosslinking between the polysaccharide chains. The crosslinked film exhibits a rough structure with multiple pores, and in this case, polymer loading and release behavior of the cell growth factor and the like may further actively occur.

The crosslinking may effectively act to incorporate and immobilize the cell growth factor in the porous film. Specifically, when the cell growth factor is negatively charged, the cell growth factor may electrostatically interact with an amine group in the porous film, and a functional group in the cell growth factor and various functional groups of the polysaccharide in the film may form hydrogen bonds. Alternatively, the cell growth factor may be further immobilized in the film by reacting with the reactive end of the crosslinking agent.

In an embodiment of the present disclosure, after the forming of the nanofilm on the surface of the cell sheet, a protective layer may be further coated to induce continuous release of the cell growth factor. The protective layer may be coated on the surface of the nanofilm to reduce the mobility of the cell growth factor so that the cell growth factor incorporated into the nanofilm may be sustainably released.

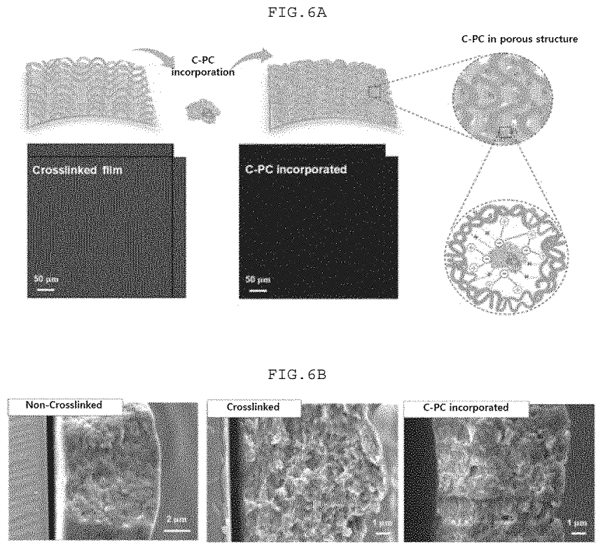

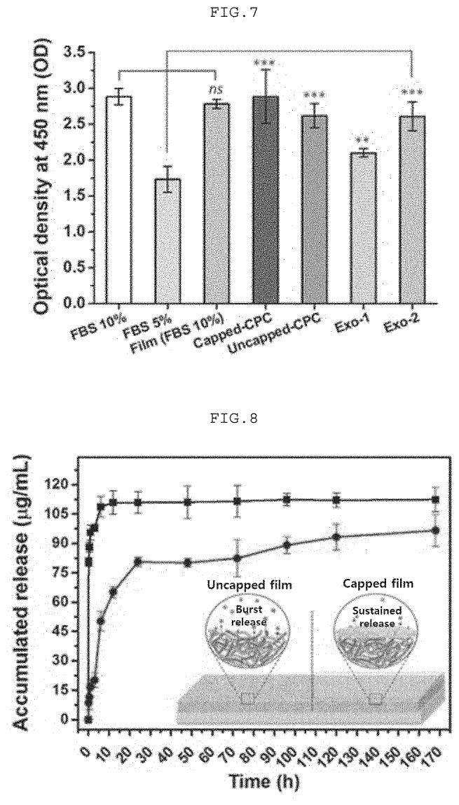

FIG. 8 illustrates a release profile of C-phycocyanin selected as a nutrient for enhancing cell proliferation. In the case of a film with a protective layer (capped film) than in the case of a film without a protective layer (uncapped film), the initial rapid diffusion was inhibited, and the sustained release behavior of C-PC was observed. The protective layer is not particularly limited, and the protective layer is preferably a sugar compound in order to increase the stability of the nutrient. As a non-limiting example, the protective layer may contain agarose.

In addition, in an embodiment of the present disclosure, the method may further include, after the forming of the nanofilm on the surface of the cell sheet, adding a cell growth factor. The cell growth factor may be incorporated inside the porous nanofilm and may be sustainably released. That is, the cell growth factor is immobilized by the electrostatic interaction or crosslinking between the positively charged material and the negatively charged material inside the porous nanofilm.

The present disclosure relates to the method for producing cultured meat, and mass proliferation of cells should be stably induced. Therefore, the method of the present disclosure may further include coating a single cell sheet with a nanofilm, and coating the nanofilm with a protective layer, thereby inducing stable mass proliferation of myoblasts. In particular, as a porous structure is formed inside the nanofilm through crosslinking and the cell growth factor and the like are incorporated and immobilized, such that stable release of the cell growth factor may be promoted, and the cell growth factor may be effectively delivered to myoblasts.

Specifically, stem cells usable for producing cultured meat are induced to proliferate and differentiate into myocytes, and the myocytes form muscle tissue. Since the quality of meat is formed by muscle movement, it is required to realize muscle tissue similar to that of a living animal. To this end, a method of continuously applying a physical stimulus to muscle fibers may be performed. In particular, in a case where the surface of the cell sheet is coated with a polymer material related to an extracellular matrix (ECM), a continuous physical stimulus may be easily transmitted between the cell sheets stacked in multiple layers, and production of proteins of the muscle fibers may be controlled. Through repeated pulling and loosening of the muscle fibers, production of collagen may be increased or decreased.

In an embodiment of the present disclosure, the inorganic material may be introduced between the negatively charged layer and the positively charged layer, if necessary. Specifically, the inorganic material may be calcium phosphate, calcium carbonate, silica, titanium oxide, or the like, but is not particularly limited thereto, and any biomineral may be used. In the case where the inorganic material is introduced, the mechanical strength of the cell sheet may be significantly improved. For example, after the negatively charged layer is stacked, in a case where an inorganic material is coated, crystallization is easily performed on the surface, and therefore, mechanical properties of the soft polymer coated on the surface of the cell sheet may be supplemented. In addition, after the multilayer cell sheet is stacked, the inorganic material is decomposed by performing a treatment with an acid when inducing mass proliferation, such that a degree of cell division may be controlled.

Specifically, stem cells differentiate into myoblasts, and the myoblasts are converted into myocytes through proliferation and differentiation processes again. According to the present disclosure, the stem cells proliferate into a large amount of myoblasts to form a cell sheet, and growth by self-organization is possible through the coating process of the cell sheet. That is, the three-dimensional cell sheet may be stably maintained by electrostatic bonding or hydrogen bonding while the coated cell sheets are stacked in multiple layers in a vertical direction without a support for cell growth.

In order to produce cultured meat for hamburger patties, sausages, and minced meat, which are mainly used without bones, a method of seeding and culturing cells on a scaffold may be used. However, in order to obtain structured meat such as steak, it is preferable to grow cells by self-organization. The self-organization refers to production of highly organized muscle tissue and cultured meat from stem cells by itself, or production of cultured meat by proliferating existing muscle tissue in a culture medium. Therefore, according to the present disclosure, structured meat may be obtained through growth by self-organization.

As the myoblasts differentiate into myocytes and the myocytes grow into muscle tissue, the muscle tissue becomes close to cultured meat, and this process may include allowing the cells to be stimulated by an ultrasonic wave, an electric current, an electromagnetic field, a magnetic field, or a combination thereof. The stimulus is a physical stimulus including a mechanical stimulus or an electrical stimulus, and an appropriate physical stimulus is applied, such that an environment similar to that in an actual body in which various stimuli such as circulatory system, nervous system, muscles, and the like exist may be created. Through this, growth promotion is induced during cell culture, and the shape, function, and development of myocytes may be controlled.

In an embodiment of the present disclosure, the method may further include adding a fat and a colorant to the muscle tissue. The fat may be added by injecting separately cultured adipocytes into the muscle tissue or adding and mixing a liquid fat with the muscle tissue when the muscle tissue is produced as a patty. This is considered one of the advantages of cultured meat because saturated fatty acids contained in meat may be replaced with beneficial fats. Since the taste of meat is derived from fat between the muscles, in order to realize the taste of cultured meat close to the taste of actual meat, vegetable fat and oils such as soybean oil, corn oil, canola oil, rice bran oil, sesame oil, extracted sesame oil, perilla oil, extracted perilla oil, safflower oil, sunflower oil, cottonseed oil, peanut oil, olive oil, palm oil, coconut oil, and red pepper seed oil, animal fats and oils such as edible beef tallow, edible lard, raw beef tallow, raw pork fat, and fish oil, and edible oil and fat processed products such as blended edible oil, flavored oil, processed oil, shortening, margarine, imitation cheese, and vegetable cream may be used.

The colorant refers to a compound that imparts color to food. In order to reproduce the red color of beef or pork, an artificial colorant, a natural colorant, natural extract (for example, beet root extract, pomegranate extract, cherry extract, carrot extract, red cabbage extract, or red seaweed extract), modified natural extract, natural juice (for example, beet root juice, pomegranate juice, cherry juice, carrot juice, red cabbage juice, or red seaweed juice), modified natural juice, food drug & cosmetics (FD&C) Red No. 3 (erythrosine), FD&C Green No. 3 (fast green FCF), FD&C Red No. 40 (allura red (AC)), FD&C Yellow No. 5 (tartazine), FD&C Yellow No. 6 (sunset yellow FCF), FD&C BLUE No. 1 (brilliant blue FCF), FD&C BLUE No. 2 (indigotine), titanium oxide, annatto, anthocyanin, betanin, β-APE 8 carotenal, β-carotene, black currant, burnt sugar, canthaxanthin, caramel, carmine/carminic acid, cochineal extract, curcumin, lutein, carotenoid, monascin, paprika, riboflavin, saffron, turmeric, and a combination thereof may be used, but are not particularly limited thereto. Additionally, a color developing agent such as nitrite, and ascorbic acid, erythorbic acid, or a salt thereof, which promotes color development of the nitrite, may be further added as a color development aid.

In addition, additionally, in order to prevent rancidity of fat, change in color, or separation of fat, an antioxidant, emulsifying salts, and the like for protein stabilization may be added. The antioxidant, the emulsifying salts, and the like may be used without limitation as long as they are widely used in the art.

In addition, the present disclosure provides cultured meat produced according to the method for producing cultured meat described above. In this case, the cultured meat may be a substitute for chicken, pork, beef, goat, lamb, duck, or fish.

In addition, the present disclosure provides a cell culture platform for producing cultured meat.

The cell culture platform for producing cultured meat includes: a substrate; a porous coating layer in which a positively charged material and a negatively charged material are alternately stacked; and a protective layer. The porous coating layer is preferably a multilayer film in which a positively charged material and a negatively charged material are alternately stacked. Specifically, the porous coating layer may be formed by crosslinking the positively charged material and the negatively charged material.

In addition, the porous coating layer may contain an active ingredient derived from microalgae therein. The active ingredient may act as a cell growth factor, and specifically, may be C-phycocyanin.

C-phycocyanin is an active ingredient extracted from cyanobacteria with a multicellular filamentous form called Spirulina platensis, and is known to have beneficial functions such as antioxidant and anti-inflammatory effects and enhancement of immune function.

In the present disclosure, the C-phycocyanin may be contained as a cell growth factor in producing cultured meat requiring mass proliferation of cells. The C-phycocyanin is contained, such that the use of animal-derived serum may be reduced, which is cost-effective, and proliferation of cells and differentiation of bone marrow hematopoietic cells may be enhanced, thereby providing an improved cell proliferation effect.

In addition, the cell culture platform for producing cultured meat according to the present disclosure may be easily applied to a cell culture plate, and provides an effect of improving proliferation of myoblasts in a serum-reduced environment during long-term culture.

Hereinafter, the method for producing cultured meat according to the present disclosure will be described in more detail with reference to Examples. However, the following Examples are only reference examples for describing the present disclosure in detail, and the present disclosure is not limited thereto and may be implemented in various forms.

### [Example 1] Production of Cell Culture Platform for Producing Cultured Meat

1-1. Production of Porous Nanofilm

A chitosan aqueous solution (CHI, medium Mw, deacetylation=75 to 85%, Sigma-Aldrich) at a concentration of 1 mg/ml and a carboxymethylcellulose sodium salt aqueous solution (CMC, Mw≈250,000, Sigma-Aldrich) at a concentration of 1 mg/ml were prepared, and the pHs of both solutions were adjusted to 4 using 1 M HCl and NaOH. Oxygen plasma-treated substrates (silicon wafer, slide glass, and OHP film) were immersed in a CHI solution for 10 minutes, and the substrates were cleaned twice with deionized water (DI water) to form a stable positively charged layer on surfaces of the substrates. Subsequently, the positively charged substrates were immersed in a negatively charged CMC solution for 10 minutes, and the substrates were cleaned in the same manner. In this process, a single bilayer (BL) film was formed on the surface of each of the substrates by the electrostatic interaction between CHI and CMC. This alternating deposition was repeated n times to produce a (CHI/CMC) film composed of n BLs.

1-2. Production of Crosslinked Porous Nanofilm

After LbL assembly, a crosslinking reaction was introduced twice to obtain a porous internal structure of a film. 1-Ethyl-3-(3-dimethylaminopropyl)carbodiimide hydrochloride (EDC, Mw≈191.71, Daejung)/N-hydroxysulfosuccinimide (NHS, Mw 115.09, Sigma-Aldrich) was used for a first crosslinking. A substrate coated with the (CHI/CMC) film was immersed in 0.1 M EDC and a 0.05 M solution of 2-(N-morpholino)ethanesulfonic acid hydrate (MES buffer, Mw 195.2, Sigma-Aldrich) supplemented with 2.5 mM NHS for 20 minutes, and then the substrate was immersed in phosphate buffered saline (1×PBS Gibco® Life Technologies) and deionized water to remove unreacted residues. For a secondary crosslinking, the substrate in which the first crosslinking was completed was incubated in a 2.5% glutaraldehyde solution (Mw 25,000, Sigma-Aldrich) for 30 minutes, and then the substrate was thoroughly cleaned with deionized water, thereby completing a crosslinked porous nanofilm (X-linked (CH I/CMC)).

1-3. Production of Cell Culture Platform for Producing Cultured Meat

A C-phycocyanin (C-PC) solution was prepared at a concentration of 0.5 mg/mL using 1×PBS as a solvent. A substrate coated with the crosslinked porous nanofilm was incubated in the C-PC solution for 12 hours at room temperature in a light-blocking environment so that C-PC was sufficiently incorporated into the film. While the film was dried, agarose was dissolved in deionized water at a concentration of 0.1 w/v %. In order to form a C-phycocyanin capping layer, an agarose solution was applied to the dried film at 25 μl per cm². The prepared film was cured at 4° C. to complete a cell culture platform (capped (CHI/CMC)/CPC) for producing cultured meat according to the present disclosure.

### [Experimental Example 1] Evaluation of Properties of Crosslinked Porous Nanofilm

1.1 FT-IR Spectral Analysis of Crosslinked Porous Nanofilm (X-Linked (CHI/CMC))

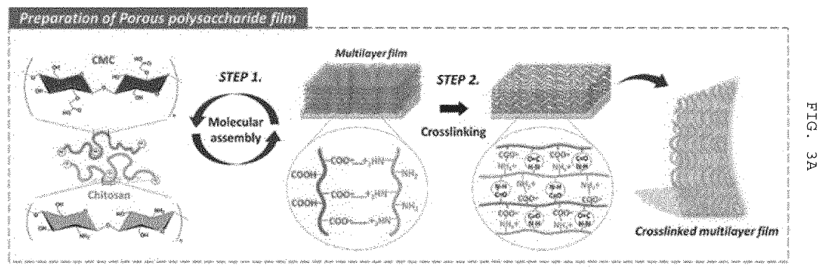

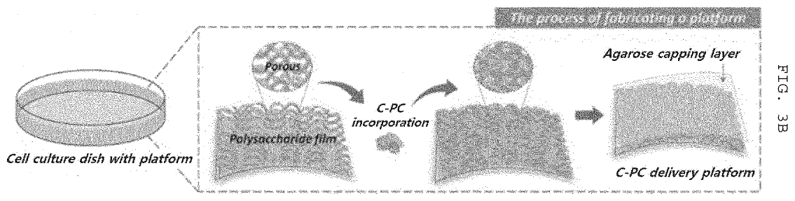

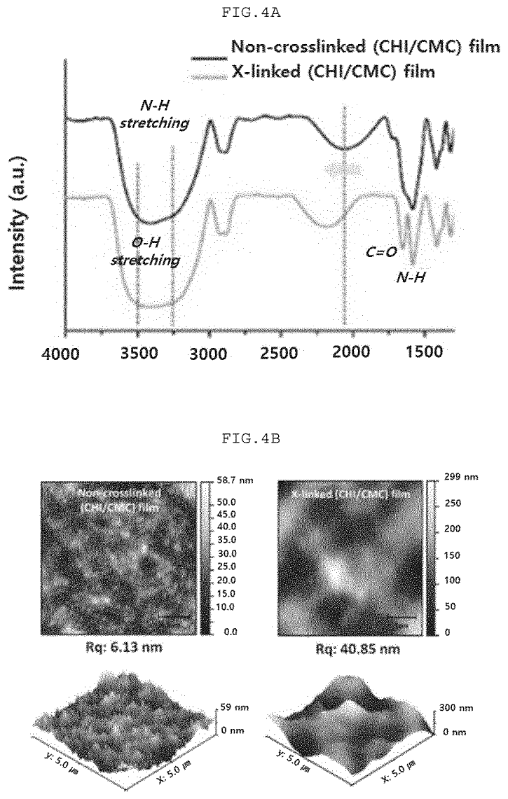

The qualitative analysis of the film before and after crosslinking and formation of additional bonds were investigated using Fourier transform infrared spectroscopy (FTIR, FT/IR-4700, Jasco, USA). The results are illustrated in FIG. 4A.

In the FT-IR spectrum of the (CHI/CMC) film, polysaccharide peaks corresponding to COC, COH, and CN were observed between 1,400 and 1,630 cm⁻¹, and overlapping peaks for OH and NH were observed between 3,200 to 3,500 cm⁻¹. In the case of the crosslinked film (X-linked (CHI/CMC)), the spectrum was similar to that of the non-crosslinked film (CHI/CMC), but O═C (1,680 cm⁻¹) and NH (1,645 cm⁻¹) bond peaks were additionally observed, and therefore, it could be confirmed that amide bonds were formed by crosslinking.

1-2. AFM Image Analysis of Crosslinked Porous Nanofilm (X-Linked (CHI/CMC))

The morphology of the film was observed by AFM and the image was analyzed using XEI and Gwyddion software. As illustrated in FIG. 4B, the (CHI/CMC) film showed a relatively dense morphology with an Rq value of 6.13 nm, whereas the crosslinked film (X-linked (CHI/CMC)) had an Rq value of 40.85 nm, which showed that a porosity was significantly increased.

### [Experimental Example 2] Evaluation of Nutrient Delivery Efficiency of Cell Culture Platform for Producing Cultured Meat

Experiment for Release of C-Phycocyanin (C-PC)

The film sample produced according to Example 1 was coated on an OHP substrate, the substrate was applied to a cell culture plate, and then cultured murine C2C12 myoblasts (passage 10) were seeded in a 12-well plate at a concentration of 8×10³ cells/well. A culture medium containing 10% FBS and a culture medium containing 5% FBS were used as positive and negative control groups, respectively, and a culture medium containing 5% FBS was used for all groups using C-PC.

\textcircled{1} A (CHI/CMC) film without C-PC, \textcircled{2} a (CHI/CMC)/CPC film group without a capping layer, \textcircled{3} a (CHI/CMC)/CPC film with a capping layer, and \textcircled{4} an exogenous C-PC group were used as experimental groups. The exogenous C-PC group was divided into two subgroups (Exo-CPC1 and Exo-CPC2), and in the case of Exo-CPC1, a medium containing C-PC was used in an amount equal to the total amount (93.22 μg/ml) of C-PC released from the capped film for 5 days. In the case of Exo-CPC2, a culture medium with daily addition of C-PC was used. At this time, the amount of C-PC added daily was calculated by dividing the total amount of C-PC released from the film by the number of days.

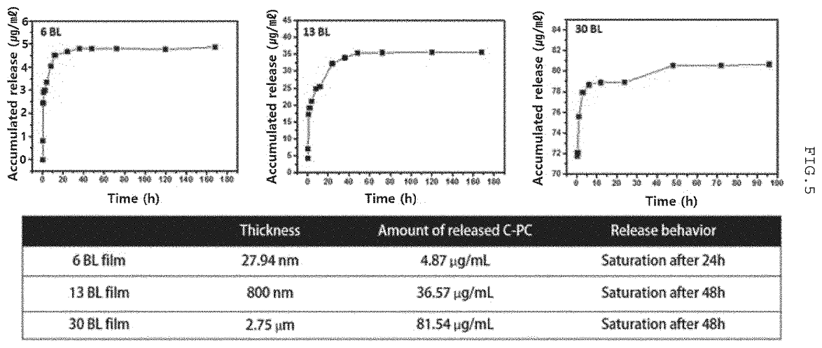

On the third day, the medium of the experimental group was replaced with a medium corresponding to each condition, and the film was not replaced. After the cells were incubated for a total of 5 days, the cells in the wells were washed with 1×PBS buffer, and the results of the cell proliferation according to each experimental group were analyzed through CCK-8 assay. As illustrated in FIG. 7, all the groups containing C-PC showed a higher degree of cell proliferation than the FBS 5% group (negative control group). It is interpreted that C-PC had a positive effect on cell proliferation by supplementing the reduced FBS. A degree of cell proliferation in \textcircled{3} the (CHI/CMC)/CPC film with a capping layer was almost similar to that of the FBS 10% group (positive control group) and was slightly higher than that of the Exo-CPC2 group, which was the highest among the experimental groups. The Exo-CPC1 group and the Exo-CPC2 group were finally treated with the same amount of C-PC, but a cell proliferation rate of the Exo-CPC2 group was significantly higher than that of the Exo-CPC1 group. These results are considered to be due to the higher activity of C-PC and the periodic stimulus applied to the cells by daily treatment of C-PC.

TABLE 1

| | Initial | FBS 10% | FBS 5% | Uncapped | Capped film | Exo-1 | Exo-2 |
|---|---|---|---|---|---|---|---|
| Number of cells | 0.8 | 19.63 ± | 3.2 ± | 15.85 ± | 19.68 ± | 8.44 ± | 15.72 ± |
| (×10⁴ cells) | | 1.61 | 2.6 | 2.36 | 5.34 | 0.83 | 2.86 |
| Expansion ratio | 1 | 24.53 | 3.99 | 19.81 | 24.60 | 10.55 | 19.63 |

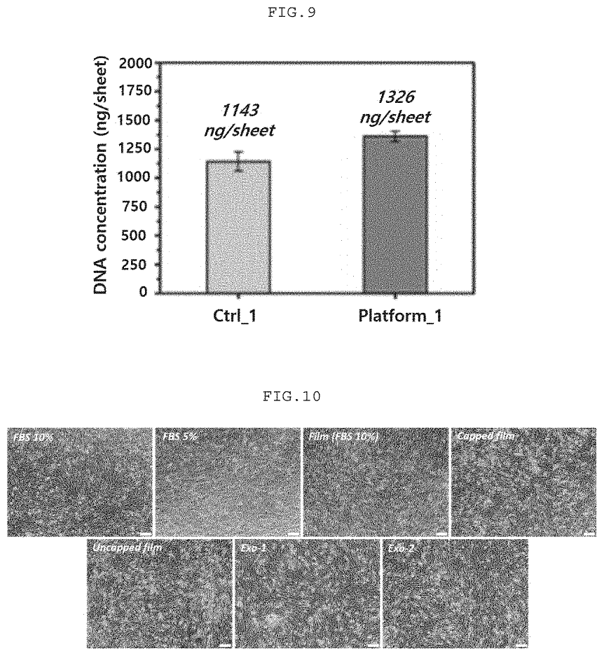

Table 1 shows the results of the number of cells and expansion ratio after culturing the cells for 5 days. In \textcircled{3} the (CHI/CMC)/CPC film with a capping layer, it was observed that the cell proliferation was about 24 times higher than the number of initially seeded cells. As can be confirmed through the optical microscope images illustrated in FIG. 10, in the case of the negative control group and the Exo-CPC1 group, the cell density was relatively lower than that in other experimental groups, and it was observed that most of the cells were present in the form of unfused myoblasts. The other groups were saturated, and the fused root form was observed.

### [Example 2] Production of Cultured Meat Obtained by Stacking Single Cell Sheets

The cell culture platform for producing cultured meat produced according to Example 1 was prepared, and 2×10⁶ cells/dish of murine C2C12 myoblasts were seeded based on a 35 mm cell culture dish. Thereafter, after the cells were cultured under conditions of 37° C. and 5% CO₂ for 12 days to form a single cell sheet, the single cell sheet was transferred to another cell sheet so that the cell sheets physically overlapped, and then, the cells were incubated under the same growth conditions for 30 hours to allow cell-to-cell junctions to proceed. Through the above process, a multilayer cell sheet was obtained.

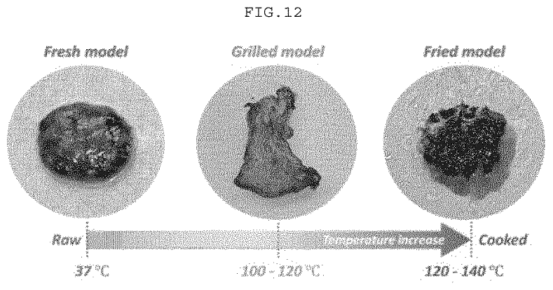

One drop of 1×PBS was added to the stacked cell sheets, and the wet sheets were stored at 4° C. for 24 hours. Thereafter, an aqueous solution of beet extract (CJ CHEILJEDANG CORP.) at a concentration of 10 mg/ml was added to the prepared cell sheets, and the cell sheets were stored at room temperature for 30 minutes. The red-dyed sheets were grilled at 100 to 120° C. and fried at 120 to 140° C. The images of the grilled and fried cultured meat models are illustrated in FIG. 12. Cultured meat before cooking was similar to raw meat, and the grilled model was similar to salami. In a case of a model fried with a lot of oil, the cultured meat was easily burned, but a shape similar to that of jerky was observed.

### [Comparative Example 1] Production of Cultured Meat Using Uncoated Cell Sheets

Cultured meat was produced in the same manner as that of Example 1, except that the coating of the surface of the cell sheet was not performed.

In the case of the uncoated cell sheet according to Comparative Example 1, cells were lost in the process of stacking the cell sheets in multiple layers to produce cultured meat, and the cell density was significantly reduced. This led to a reduction in differentiation efficiency into muscle tissue, and it was confirmed that cultured meat was not properly formed. In the cases of the cultured meat produced according to Examples 1 to 3 in which the cell sheet was coated, it was observed that the cells differentiated into hard muscle tissue, the fat was inserted between the muscles, and the cultured meat before mincing was visually similar to the actual meat tissue.

### [Experimental Example 3] Evaluation of Mechanical Strength of Cell Sheet

According to Example 1 and Comparative Example 1, the mechanical strength of each of the cell sheet coated with the positively charged layer/negatively charged layer once (2-layer), the cell sheet coated with the positively charged layer/negatively charged layer twice (4-layer), the cell sheet coated with the positively charged layer/negatively charged layer three times (6-layer), and the uncoated cell sheet (control) was evaluated.

The strength of the cell sheet was evaluated with a compression analyzer designed for testing a compressive strength of a micro-smooth material. The indenter was equipped with a flat cylindrical stainless steel probe with a load cell of 4.9 N and a diameter of 3 mm. A force was applied vertically to the cell sheet and a measurement speed was set to 10 μm/sec. A depth of less than 4.9 mN was regarded as the initial depth (L0) of the cell sheet, and a depth when the measuring tip reached the plate dish was regarded as the total length (Lt) of the sheet. A length L of the cell sheet was calculated as a difference between Lt and L0, and each measured length was normalized to L. The mechanical properties of the cell sheet were obtained from the compressive force (N) applied to each point of the cell sheet. A coefficient of compressibility was calibrated using a stress-strain curve and load-displacement data obtained from indentation measurements were used to obtain a modulus by Oliver/Pharr mathematical model.

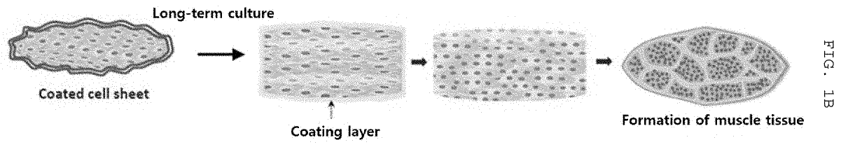

FIG. 2 illustrates the results of evaluating the mechanical strength. It can be confirmed that the mechanical strength of the cell sheet is improved when the cell sheet is coated in multiple layers compared to the uncoated cell sheet. The 4-layer and the 6-layer show that the strength is significantly increased compared to the case of coating the surface of the sheet with the 2-layer.

### [Experimental Example 4] Evaluation of Density and Differentiation of Cell Sheet

In order to compare the cell density and cell function in the uncoated cell sheet and the coated cell sheet, H&E staining of each of the cell sheets was analyzed and the density of each of cell sheets was quantified. First, an OHP substrate coated with a capped (CHI/CMC) CPC film was introduced into the wall of the cell culture plate, and then a single cell sheet was prepared. After cells were cultured for 10 days, Haematoxylin and Eosin staining (H&E staining) was performed on the single layer or 4-layer cell sheet, and DNA quantification was analyzed (Quant-iT™ PicoGreen dsDNA assay kit, Invitrogen).

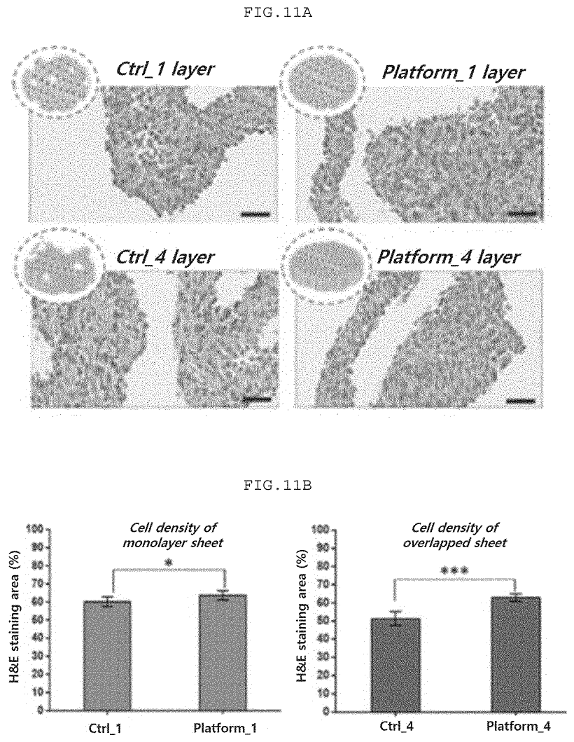

The H&E staining image is illustrated in FIG. 11A. Haematoxylin stains the nucleus, and eosin stains the cytoplasm. The more stained areas and fewer empty spaces in the cell sheet, the higher the density of the cell sheet. The density of the cell sheet was quantified by calculating the percentage of stained area in an area of 180 μm×180 μm of each sample. Both the single layer and 4-layer cell sheets showed higher staining levels in the group using the C-PC delivery platform compared to the control group. These results suggest that the nutritional factors released from the platform continuously provide nutrients and suppress cell aging and cell death.

A cell sheet prepared for DNA quantification was lysed in 200 μ/ml of lysate buffer consisting of 0.5 mg/ml of Proteinase K, EDTA solution (5 mM EDTA in 1×PBS) and 0.1% Triton-X for 3 hours, a working solution (reagent:buffer=1:200) was applied to the standard and the lysed cell sheet solution. The sample was analyzed using a microplate reader (SpectraMax ABS, Molecular Devices). The results are illustrated in FIG. 7. It could be confirmed that 1,143 and 1,326 ng of DNA were contained in the cell sheets of the control group and the group to which the cell culture platform was applied, respectively, which showed that a higher amount of DNA was present in the cell sheet of the group to which the cell culture platform was applied.

Although preferred embodiments of the present disclosure have been described, the present disclosure is not limited to the embodiments, but may be implemented in various modifications within the scope of the claims, the description of the invention, and the accompanying drawings. These modifications also fall within the scope of the present disclosure.

1. A method for producing cultured meat, comprising:
forming a single cell sheet by culturing cells usable for producing cultured meat;
obtaining the single cell sheet;
forming a nanofilm on a surface of the cell sheet by coating the obtained single cell sheet;
forming a multilayer cell sheet by stacking the coated single cell sheets; and
forming muscle tissue from the stacked cell sheets.

2. The method of claim 1, wherein the cells usable for producing cultured meat are mesenchymal stem cells (MSCs), induced pluripotent stem cells (iPSCs), satellite cells, adipocytes, or embryonic stem cells.

3. The method of claim 1, wherein the coating is performed to form a multilayer nanofilm using one or two or more selected from the group consisting of electrostatic attraction, van der Waals force, hydrophobic bonding, hydrogen bonding, and covalent bonding.

4. The method of claim 1, wherein the nanofilm is formed by alternately stacking a positively charged material and a negatively charged material.

5. The method of claim 4, wherein the positively charged material is one or two or more selected from the group consisting of chitosan, starch, collagen, gelatin, fibrinogen, silk fibroin, casein, elastin, laminin, and fibronectin.

6. The method of claim 4, wherein the negatively charged material is one or two or more selected from the group consisting of hyaluronic acid, alginate, tannic acid, lignin, cellulose, heparin, carrageenan, agar, xanthan gum, gum arabic, glucomannan, carboxymethylcellulose (CMC), and tara gum.

7. The method of claim 1, wherein a thickness of the nanofilm is 50 to 5,000 nm.

8. The method of claim 1, further comprising, after the forming of the nanofilm, forming a protective layer.

9. The method of claim 1, wherein in the culturing of the coated cells, the coated cells are stimulated by an ultrasonic wave, an electric current, an electromagnetic field, a magnetic field, or a combination thereof.

10. The method of claim 1, further comprising, after the forming of the nanofilm, adding a cell growth factor.

11. The method of claim 1, further comprising adding a fat and a colorant to the muscle tissue.

12. Cultured meat produced by the method for producing cultured meat of claim 1.

13. A cell culture platform for producing cultured meat, comprising:
a substrate;
a porous coating layer in which a positively charged material and a negatively charged material are alternately stacked; and
a protective layer.

14. The cell culture platform of claim 13, wherein the porous coating layer is formed by crosslinking the positively charged material and the negatively charged material.

15. The cell culture platform of claim 13, wherein the porous coating layer contains C-phycocyanin.
